(19) United States
(12) Patent Application Publication
(10) Pub. No.: US 2018/0272336 A1
(43) Pub. Date: Sep. 27, 2018

(54) SYSTEM AND METHOD FOR STRIPPING A SAMPLE OF CHEMICAL COMPOUNDS

(71) Applicants: Wesley Kimbell, Tomball, TX (US); David Flower, Tomball, TX (US)

(72) Inventors: Wesley Kimbell, Tomball, TX (US); David Flower, Tomball, TX (US)

(21) Appl. No.: 15/464,661

(22) Filed: Mar. 21, 2017

(51) Int. Cl. B01L 3/00; G01N 33/00

(52) U.S. Cl. CPC B01L 3/502; B01L 2300/0681; G01N 33/0016

(57) ABSTRACT

A device for testing the constituent elements of a fluid compound is provided. The device includes a hollow chamber that is filled with a sample fluid. At one end of the hallow chamber, a transfer fluid (such as air) is pumped through a permeable membrane. A portion of the sample fluid permeates the permeable membrane and is absorbed into the transfer fluid. The transfer fluid, with the absorbed sample fluid now in gaseous phase, is sent to a testing device in order to determine the constituent elements of the sample fluid.

### CROSS REFERENCE TO RELATED APPLICATION

This application is a conversion of and claims priority from U.S. Provisional Patent Application No. 62/311,858 which was filed on Mar. 22, 2016 by the same inventors as the present application and is incorporated herein by reference for all purposes.

### FIELD OF THE INVENTION

The present invention relates to the sampling of chemical compounds. More particularly, the present invention relates to a system and method for creating a vapor from a sample fluid that maintains the compositional representation of the sample fluid so that the vapor can be fed to a detector that can only accept gaseous samples.

### BACKGROUND OF THE INVENTION

A prior art design is illustrated in FIG. 1. The prior art design consisted of a hollow housing 10, containing four fitting ports 60, 61, 62 and 63 for entry and exit of a liquid or carrier gas, two (60 and 61) at the top end 14 of the enclosed housing 10 and two (62 and 63) at the bottom end 12 of the enclosed housing 10. Connecting the two fitting ports 62 and 63 on the bottom end 12 is a permeable polymer membrane 20 with each end of the permeable polymer membrane 20 attached to the respective post tubes 42 and 43 via membrane sleeves 42 and 43, respectively as shown in FIG. 1. Post tubes 42 and 43 connect the membrane sleeves 42 and 43 to the fitting ports 62 and 63, respectively.

In operation, a test fluid would be injected into port 62 and the test fluid that was not otherwise vaporized would exit the housing 10 at fitting port 63. In other words, the sample fluid travels the inside of the permeable membrane tube 20 and then the sample fluid exits the other port to drain. The permeable membrane tube 20 allows a compositional representation of the sample fluid to be vaporized and thus permeate/dissolve through the membrane tubing 20 to the outer side of the membrane tube 20 and into the hollow interior 16 of the housing 10.

Any and all volatile organic compounds and other components including hydrogen sulfide will move through the membrane 20 driven by, and proportional to, the partial pressure of each component (e.g., volatile hydrocarbons, H₂S, etc.). Each constituent component of the fluid sample can make the partial pressure change on the membrane surface. Concurrently, a clean carrier air/gas enters the fitting port 60 on the top side 14 of the housing 10 and into the hollow interior 16 and sweeps the permeated/dissolved compositional vapor of the sample fluid to the exhaust port fitting 61 to a detector, sensor, or other apparatus for use (not shown). The carrier gas/air keeps the partial pressure at essentially zero on the outer side of the membrane 20.

Several issues exist with the prior art device. First, due to the small internal diameter of the permeable membrane tube 20, the flow rate of the sample fluid must be regulated to a modest amount in order to increase time available in the membrane 20, which negatively affects user experience because it takes longer to get a requisite amount of vapor. Furthermore, the small internal diameter of the membrane tubing limits liquids to only low viscosity due to the inability for higher viscosity liquids of to squeeze through the membrane 20.

There is, therefore, a need in the art both to decrease the time needed to sample a fluid, and to enable testing of fluids with higher viscosity.

### BRIEF SUMMARY OF THE INVENTION

The present invention includes a hollow chamber that is filled with a sample fluid. At one end of the hallow chamber, a transfer fluid (such as air) is pumped through a permeable membrane. A portion of the sample fluid permeates the permeable membrane and is absorbed into the transfer fluid. The transfer fluid, with the absorbed sample fluid now in gaseous phase, is sent to a testing device in order to determine the constituent elements of the sample fluid.

### BRIEF DESCRIPTION OF THE DRAWINGS

FIG. 1 illustrates a prior art device.

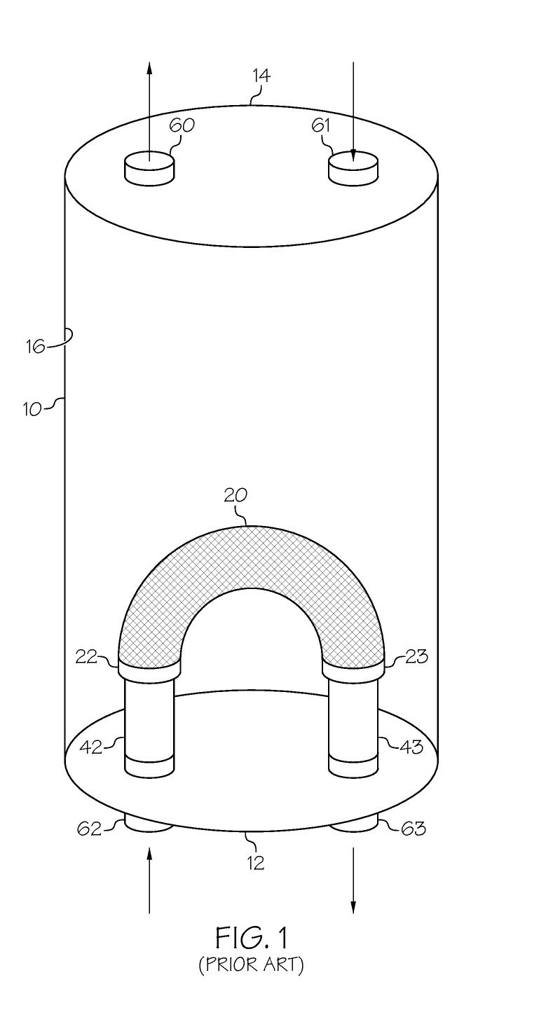

FIG. 2 illustrates a frontal view of one embodiment of the present invention.

FIG. 3 is a chart illustrating the decrease in response time afforded by the present invention.

FIG. 4 illustrates a frontal view of an alternate embodiment of the present invention.

### DETAILED DESCRIPTION OF THE INVENTION

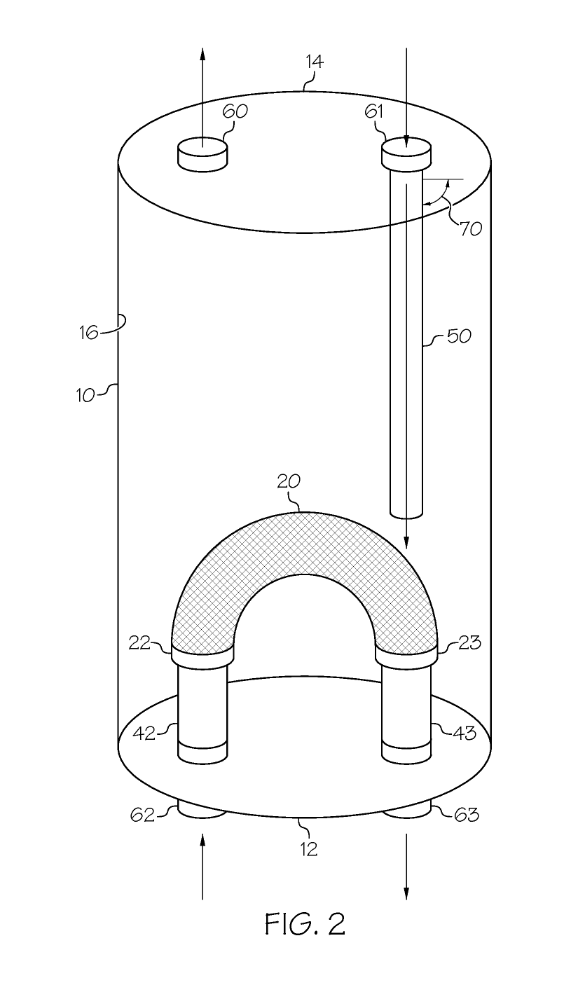

To solve the issues associated with the prior art, the present invention reverses the operational theory of the prior art devices and adds a component and operational variation to achieve results that both surprise and yield additional functionality. Referring to FIG. 2, the sample fluid flows in at port 61 and out at port 60 at the top end 14 of the container 10. The permeable membrane 20 is situated at the bottom end 12 of the container 10. The two ends 22 and 23 of the permeable membrane 20 are connected to posts 42 and 43, respectively, which are in turn connected to ports 62 and 63, respectively. The carrier (transfer) air/gas flows through the interior of the permeable membrane 20 from port 62 and to port 63 as illustrated in FIG. 2. The compositional vapor of the sample fluid, therefore, permeates into the inside of the permeable membrane 20, enabling the carrier air/gas to sweep the permeated compositional vapor from the sample fluid out the port 63 and to a detector, sensor, or other apparatus for measurement or other use.

Importantly, a directional dip tube 50 is attached to the port 61 at the top end 14 of the container 10 so that the sample fluid is directed at a precise angle onto and across the membrane 20 that ensures turbulent flow (as opposed the less efficient laminar flow) across the membrane 20, thereby optimizing the efficiently and speed of permeation. The angle of direction 70 of the dip tube 50 can be adjusted to account for different sample fluids and/or carrier fluids and/or variations in the permeability of the membrane 20.

In an alternate embodiment of the present invention, the real-time measurement of the detected gas can be used to alter the angle 70 or the flow rate of the sample fluid within the housing 10 to optimize the speed and/or flow rate of the sampling. Indeed, the flow rate of the carrier fluid is important because it needs to be constant so that no dilution occurs. The arrangement of the present invention as illustrated in FIG. 2, including the carrier fluid within the permeable membrane 20 and the sample fluid directed by the dip tube 50 (to optimize the angle if incidence of the sample fluid onto the permeable membrane 20) is what enables a faster response time even though the flow rate of the sample fluid is increased dramatically.

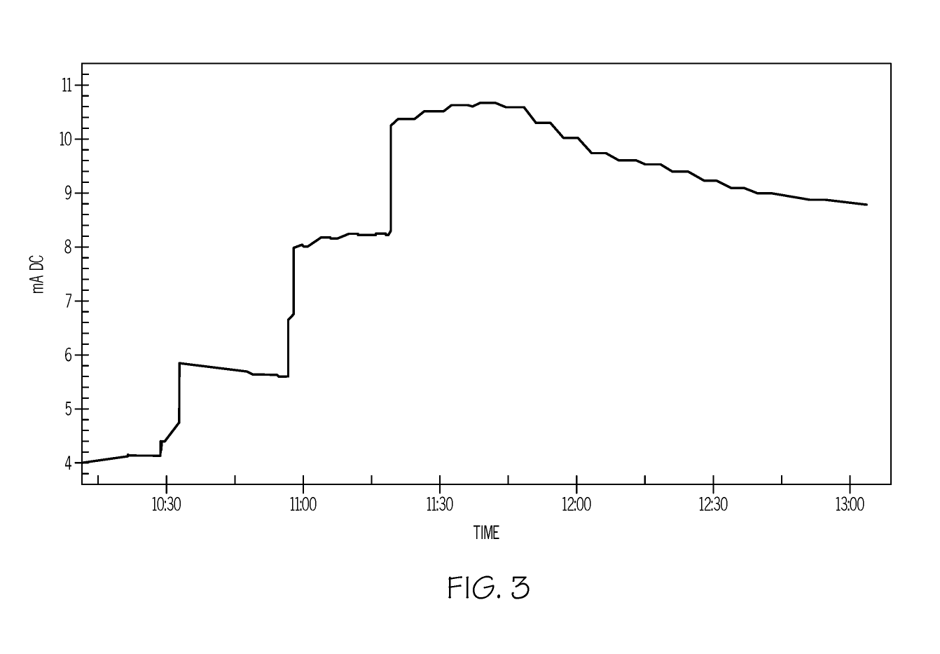

In yet another embodiment of the present invention, the housing 10 may also be adjusted in shape and/or size to increase velocity of sample fluid and its injection direction so that permeation through the membrane 20 is further optimized for efficiency and speed. Increasing the flow rate of the sample fluid increases the rate of permeation (speed and efficiency) of the vapor through the membrane 20. The data shown in FIG. 3 illustrates the decrease in response time (lag) with faster flow rates that the prior art device cannot accomplish. In one embodiment of the present invention, rotameters and/or flow meters are used to adjust the flow rates of the carrier fluid and the sample fluid.

The following is an example of the operation of the present invention that generated the data illustrated in FIG. 3. Other examples with the present invention are, of course, quite possible. In this case, the present invention was connected to an analyzer that quantifies hydrogen sulfide in a crude oil sample. The analyzer was calibrated to be approximately 25 PPMv at full scale. A series of three crude oil samples were prepared in sample containers with six PPMv of H₂S by volumetrically blending pure H₂S into the crude oil containers. The sample had a viscosity of approximately 30 cP. The flow rate of the sample fluid was approximately 200 ml/min. The first H₂S sample was introduced at 10:28 as illustrated in FIG. 3. The graph in FIG. 3 shows 100% of response at 10:33, approximately 5 minutes later. The initial response was realized in just a few seconds. This was a substantial departure from what was realized with the prior art device.

A second H₂S sample was introduced at 10:56. The graph of FIG. 3 shows 100% of the response was realized at 10:58, approximately 2 minutes after introduction. Again, the present invention yielded substantially reduced response (lag) times.

A third H₂S sample was introduced at 11:17, and the graph of FIG. 3 shows 100% of response was achieved at 11:19, within approximately 2 minutes.

The prior art device could not attain a flow rate much above approximate 60 ml/min. without causing damage to the membrane 20. In stark contrast, the present invention with the variable dip tube 50 was able to achieve reliable detection in much shorter time spans, in many cases less than half of that of the prior art device.

In another embodiment, the present invention may act as a filter, due to the membrane 20 effectively blocking undesirable amounts of mists, liquids, vapors, water, or other elements that are entrained in sample fluid. Using the present invention in this way results in a filtered, ultra-clean/pure representative and proportional sample that permeates/dissolves/vaporizes through the membrane 20 for use elsewhere. This is beneficial because gas-only analyzers cannot accept mists, vapors, water. They can accept only clean and dry sample gas. Moreover, most of the stripped gas will be the carrier air with a proportional and small amount of the component of interest still permeating through and being representative/proportional of the sample fluid which may prove useful for a variety of activities.

In yet another embodiment, the present invention may act as a concentrator or dilutor, based upon the adjustment of the membrane 20 to suit the user's needs to concentrate or dilute the stripped sample with the carrier air fluid. This embodiment can be useful when the component of interest that is to be measured is of such a low concentration that the attendant analyzer/sensor cannot detect the presence of the component of interest. The membrane 20 can be adjusted (usually lengthened) to proportionally concentrate the component of interest so that said component can be detected/analyzed by the analyzer/sensor. Alternatively, when the sample component of interested is of too high a concentration for the analyzer/sensor to accept, the membrane 20 can be adjusted (usually shortened) which results in the component of interested being representatively and proportionally diluted so that the analyzer/sensor may be able to measure the component of interest.

In yet another embodiment, the system of the present invention may act as a range concentrator for the purposes of calibrating/validating analyzers/sensors by permeating a known concentration of components to the sensor/detector that is to be calibrated. An example of this embodiment would work by flowing a pure concentration (99% or higher) of the component(s) of interest across the membrane 20 while the carrier fluid sweeps a diluted concentration to the analyze/sensor for calibration. The membrane would be adjusted (usually lengthened) based upon a calculation that is determined with one or more of the following factors: flow rate of the calibration component, the flow rate of carrier fluid, the concentration of the calibration component(s), the desired component concentration for calibration, the temperature of the calibration component, the temperature of membrane 20, and the temperature of whole invention. In this embodiment of the present invention, one or more temperature sensors would be utilized in appropriate places within the housing 10. However, it is often more convenient and helpful to put the entire container 10 in a temperature-controlled environment so that all the temperatures can be changed in unison. In alternate embodiments of the present invention, it may be helpful to adjust the temperature. of the membrane 20 and the internal housing 16 separately, which is useful to prevent condensation from “breaking through” the membrane 20. That embodiment would entail keeping the membrane 20 cooler than the internal housing 16 to prevent condensation.

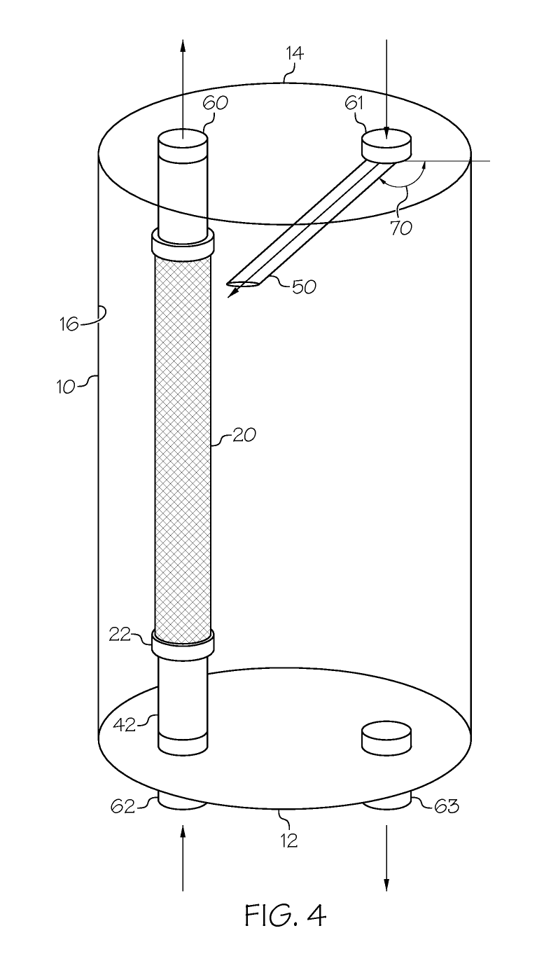

FIG. 4 illustrates an alternate embodiment of the present invention. Referring to FIG. 4, the permeable membrane 20 is fitted colinearly within the hollow portion 16 of the housing 10. In this alternate embodiment, the carrier fluid is injected at the bottom side 12 of the housing 10 through port 62, and is exhausted out port 60 on the top side 14 of the housing 10. In this embodiment, the dip tube 50 is positioned (variably) at angle 70 so that the sample fluid that is injected into port 61 is directed to the permeable membrane 20 as illustrated in FIG. 4 and eventually exhausted out port 63. As before, the angle 70, and the length of the membrane 20 can be adjusted for the various fluids (both carrier and sample) to suit the testing scenario. Other embodiments with different configurations for the permeable membrane 20, such as an “S” curve between two diagonally opposing ports is also possible.

While the present invention, as to its objects and advantages, has been described herein as carried out in specific embodiments thereof, it is not desired to be limited thereby but it is intended to cover the invention broadly within the spirit and scope of the accompanying claims.

What is claimed is:

1. A container, the container having a top end and a bottom end, the container further comprising:
a hollow portion that is constructed and arranged to contain a sample fluid;
two ports at the top end of the container, the two ports at the top end constructed and arranged to enable the flow of the sample fluid through the hollow portion of the container;
two ports at the bottom end of the container, the two ports at the bottom end of the container connected to each other with a permeable membrane that is situated within the hollow portion of the container and is constructed and arranged to enable the flow of a transfer fluid between the two ports at the bottom end of the container within the permeable membrane; and
a dip tube, the dip tube attached to one of the ports at the top end of the container, the dip tube constructed and arranged to force the sample fluid into close proximity of the permeable membrane;
wherein a constituent portion of the sample fluid is vaporized and permeates the permeable membrane to be carried off by the transfer fluid to a detector.

2. The container of claim 1, wherein a distance between the dip tube and the permeable membrane is variable.

3. The container of claim 1, wherein the flow rate of the carrier fluid is constant at the time of measurement.

4. The container of claim 1, wherein the flow across the permeable membrane is turbulent.

5. The container of claim 1, wherein the flow rate of the carrier fluid is variable.

6. The container of claim 1, wherein the flow rate of the sample fluid is variable.

7. The container of claim 1, wherein the proximity and angle of the dip tube can be modified to account for different fluids.

8. The container of claim 1, wherein the permeability of the permeable membrane can be adjusted.

9. The container of claim 1, wherein the sample fluid, the carrier fluid and the permeability of the permeable membrane can be adjusted.

10. A container, the container having a top end and a bottom end, the container further comprising:
a hollow portion that is constructed and arranged to contain a sample fluid;
two ports at the top end of the container, the two ports at the top end constructed and arranged to enable the flow of the sample fluid through the hollow portion of the container;
one port at the bottom end of the container, the port at the bottom end of the container connected to one of the top ports with a permeable membrane that is situated within the hollow portion of the container and is constructed and arranged to enable the flow of a transfer fluid between the port at the bottom end of the container and one of the ports at the top end of the container within the permeable membrane; and
a dip tube, the dip tube attached to one of the ports at the top end of the container, the dip tube constructed and arranged to force the sample fluid into close proximity of the permeable membrane;
wherein a constituent portion of the sample fluid is vaporized and permeates the permeable membrane to be carried off by the transfer fluid to a detector.

11. The container of claim 10, wherein a distance between the dip tube and the permeable membrane is variable.

12. The container of claim 10, wherein the flow rate of the carrier fluid is constant at the time of measurement.

13. The container of claim 10, wherein the flow across the permeable membrane is turbulent.

14. The container of claim 10, wherein the flow rate of the carrier fluid is variable.

15. The container of claim 10, wherein the flow rate of the sample fluid is variable.

16. The container of claim 10, wherein the proximity and angle of the dip tube can be modified to account for different fluids.

17. The container of claim 10, wherein the permeability of the permeable membrane can be adjusted.

18. The container of claim 10, wherein the sample fluid, the carrier fluid and the permeability of the permeable membrane can be adjusted.

19. A method for determining the contents of a fluid comprising:
providing a hollow cylinder into which a carrier fluid can be placed;
providing sample tube within the cylinder, the sample tube having a permeable membrane, the sample tube further having a sample fluid that can permeate through the permeable membrane;
providing a dip tube, the dip tube constructed and arranged to extract the sample fluid that permeates through the permeable membrane out of the cylinder;
wherein the extracted sample fluid can be sent to an analyzer.
